(19) United States
(12) Patent Application Publication
(10) Pub. No.: US 2009/0082371 A1
(43) Pub. Date: Mar. 26, 2009

(54) Treatment of Viral Disease and Cancer With Nf-kappaB Inhibitors

(76) Inventor: Zhimin Lu, Pearland, TX (US)

Correspondence Address: ROBERT E. BUSHNELL & LAW FIRM, 2029 K STREET NW, SUITE 600, WASHINGTON, DC, 20006-1004, US

(21) Appl. No.: 11/922,323

(22) PCT Filed: Jun. 19, 2006
(86) PCT No.: PCT/CN2006/001383

§ 371 Date: Dec. 17, 2007

(30) Foreign Application Priority Data
Jun. 17, 2005 (CN) 2005-10077504.4

(51) Int. Cl. A61K 31/517; A61K 31/343; A61K 31/10; A61K 31/35; A61K 31/12

(52) U.S. Cl. 514/266.4; 514/470; 514/710; 514/456; 514/683

(57) ABSTRACT

A method using quinazoline derivatives, celastrol, cape, BAY 11-7082, rocaglamide, and 7-Methoxy-5,11,12-trihydroxy-coumestan is administered to a mammal for the treatment and prevention of viral diseases and cancers. The chemical compounds are targeted to inhibit NF-κB transcriptional activity. These treatments for viral diseases, especially for infection caused by HIV, and cancers may be accomplished utilizing quinazoline derivatives, celastrol, cape, BAY 11-7082, rocaglamide, 7-Methoxy-5,11,12-trihydroxy-coumestan, and compounds similar to them alone or in combination with prior art other therapy.

### CLAIM OF PRIORITY

This application makes reference to, incorporates the same herein, and claims all benefits accruing under Title 35 U.S. Code §365(b)(c) of my PCT International application entitled USE OF NF-κB INHIBITOR FOR TREATING SEVERAL DISEASES, filed on 19 Jun. 2006 and duly assigned Serial No. PCT/CN2006/001383.

### FIELD OF THE INVENTION

The present invention relates generally methods for treating and preventing viral diseases and cancer with quinazoline derivatives, celastrol, cape, BAY 11-7082, rocaglamide, and 7-Methoxy-5,11,12-trihydroxy-coumestan. More specially, it relates to methods for treating and preventing diseases caused by retroviruses including HIV and human cancers.

### BACKGROUND OF THE INVENTION

The nuclear factor kappa B (NF-κB) is a pivotal transcription factor that plays important roles in regulating gene expression in immune system as well as other non-immune system. NF-κB is activated and regulates the expression of cytokines, growth factors, and effector enzymes in response to ligation of many receptors including T-cell receptors, B-cell receptors, tumor necrosis factor alpha receptor, CD40, BAFF-BAFF receptor, lymphotoxin-beta receptor, and the Toll/interleukin-1 receptor. The regulation of NF-κB activity occurs at several levels including controlled cytoplasmic-nuclear shuttling and modulation of its transcriptional activity. Controlled activation of NF-κB is essential for normal innate and adaptive immune responses, and dysregulated NF-κB signaling in lymphocytes contributes to diseases including chronic inflammation and autoimmunity.

Although the role of NF-κB in immune, inflammatory, and apoptotic response has been documented, whether NF-κB can be an effective molecular target for treatment of viral diseases and cancer is not known. Experiments in this invention using NF-κB inhibitors, such as quinazoline derivatives, celastrol, cape, and rocaglamide, have shown that these compounds are effective for inhibition of infection and replication retrovirus, especially human immunodeficiency virus (HIV). These NF-κB inhibitors are also effective for the treatment of breast cancer, leukemia, lymphoma, lung cancer, skin cancer, prostate cancer, liver cancer, brain tumor, cervical cancer, pancreatic cancer, and cancers located in the digestive tract including gastric cancer and colon cancers.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings in which like reference symbols indicate the same or similar components, wherein:

FIG. 1 shows examples of treatment of viral disease and cancer with NF-κB inhibitors.

FIG. 2 shows examples of the effect of NF-κB inhibitors on HIV infection.

### DETAILED DESCRIPTION OF PREFERRED EMBODIMENTS

### Composition

The present invention relates the application of NF-κB inhibitors such as 6-aminoquinazoline derivatives (To be M et al., Bioorg Med Chem. 2003; 11:383-391. Tobe M, et al., Bioorg Med Chem. 2003; 11:3869-3878), celastrol (Jin H Z, et al., J Nat Prod. 2002; 65:89-91), cape (Natarajan K, et al., Proc Natl Acad Sci USA. 1996; 93: 9090-9095), BAY 11-7082 (Izban K F, et al., Hum Pathol. 2000; 31:1482-1490), rocaglamide (Baumann B, et al., J Biol Chem. 2002; 277:44791-800), and 7-Methoxy-5,11,12-trihydroxy-coumestan (Kobori M, et al., Cell Death Differ. 2004; 11:123-130) for treatment of viral diseases and cancer.

The quinazoline derivatives are compounds having the following formula or its pharmaceutically acceptable salt as an active ingredient.

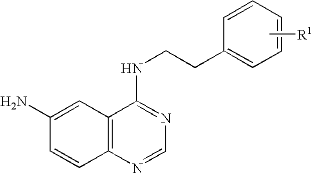

R¹ represents OR², a substituted or unsubstituted aryl, a substituted or unsubstituted heteroaryl, a substituted or unsubstituted alkyl, a substituted or unsubstituted alkenyl, a substituted or unsubstituted alkynyl, the phenooxy group, n-butoxy, n-pentyloxy, n-propoxy, iso-propyl, n-hexyloxy analogues, benzyloxy, pyridylmethoxy, quinolinylmethoxy, allyloxy, propargyloxy, or ethoxy. R² is a substituted or un-substituted alkyl with 1 to 6 carbon, a substituted or un-substituted alkenyl with 2 to 6 carbon, a substituted or un-substituted alkynyl with 2 to 6 carbon, a substituted or un-substituted aryl, substituted or non-substituted heteroaryl, a substituted or un-substituted aralkyl, or a substituted or un-substituted heteroarylalkyl.

Preferred chemicals for use in accordance with the present invention are:

1. 6-Amino-4-(4-phenoxy)phenethylaminoquinazoline.

2. 6-Amino-4-(4-n-pentyloxy)phenethylaminoquinazoline.

3. 6-Amino-4-(4-n-hexyloxy)phenethylaminoquinazoline.

4. 6-Amino-4-(4-n-butoxy)phenethylaminoquinazoline.

5. 6-Amino-4-(4-benzyloxy)phenethylaminoquinazoline.

6. 6-Amino-4-[4-(2-pyridylmethyloxy)phenethylaminoquinazoline.

7. 4-(4-Allyloxy)phenethylamino-6-aminoquinazoline.

8. 6-Amino-4-(4-propargyloxy)phenethylaminoquinazoline.

9. 6-Amino-4-(4-ethoxy)phenethylaminoquinazoline.

10. 6-Amino-4-(4-propoxy)phenethylaminoquinazoline.

11. 6-Amino-4-(4-iso-propoxy)phenethylaminoquinazoline.

The rocaglamide derivatives are compounds having the structure similar to the following formula or its pharmaceutically acceptable salt as an active ingredient.

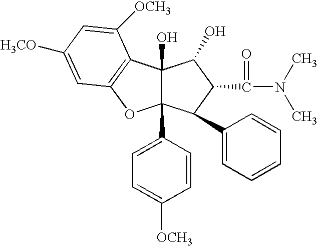

Practice of the invention will be more understood from the following examples, which are presented herein for illustration only and should not be considered as limiting the invention in any way.

### EXAMPLES

### A. The Effect of NF-κB Inhibitors on Viral Infection

### A). The Effect of NF-κB Inhibitors on Retroviral Infection.

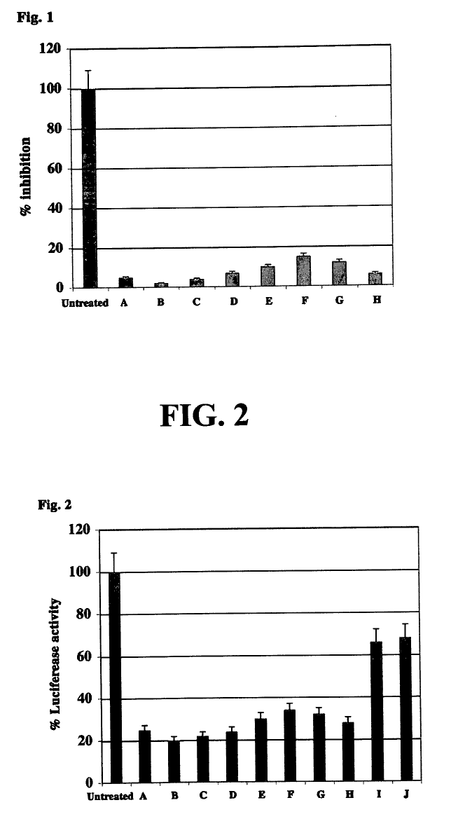

FIG. 1. Retroviruses expressing a marker protein, the green fluorescent protein (GFP), were prepared as described previously (Lu M. and Shenk T., J Virol. 1999; 73: 676-683). 293T human embryonic kidney cells were infected with the GFP-retrovirus. Some cells were pretreated with 6-Amino-4-(4-phenoxy)phenethylaminoquinazoline (Column A), 6-Amino-4-(4-n-pentyloxy)phenethylaminoquinazoline (Column B), 6-Amino-4-(4-n-hexyloxy)phenethylaminoquinazoline (Column C), 6-Amino-4-(4-n-butoxy)phenethylaminoquinazoline (Column D), 6-Amino-4-(4-benzyloxy)phenethylaminoquinazoline (Column E), 6-Amino-4-[4-(2-pyridylmethyloxy)phenethylaminoquinazoline (Column F), 4-(4-Allyloxy)phenethylamino-6-aminoquinazoline (Column G), or rocaglamide (Column H) for 30 mins before viral infection. Two days later, the cells were photographed with a digital camera mounted on a microscope. There were no obvious cell deaths observed with the tested dosages. Data represent the mean±standard deviation of three independent experiments.

The experimental results were illustrated further as below.

### Example 1

### Inhibition of Retroviral Infection and Replication by 6-Amino-4-(4-phenoxy)phenethylaminoquinazoline

293T cells were pretreated with or without 6-Amino-4-(4-phenoxy)phenethylaminoquinazoline (1.5 μM) 30 mins before infection of retrovirus expressing GFP. Two days later, the number of infected cells from two separate experiments was counted. The quantitative data showed that the cells treated with 6-Amino-4-(4-phenoxy)phenethylaminoquinazoline displayed about 95% inhibition of viral infection and replication compared to untreated cells (FIG. 1. Column A).

### Example 2

### Inhibition of Retroviral Infection and Replication by 6-Amino-4-(4-n-pentyloxy)phenethylaminoquinazoline

293T cells were pretreated with or without 6-Amino-4-(4-n-pentyloxy)phenethylaminoquinazoline (1.5 μM) 30 mins before infection of retrovirus expressing GFP. Two days later, the number of infected cells from two separate experiments was counted. The quantitative data showed that 6-Amino-4-(4-phenoxy)phenethylaminoquinazoline inhibited about 98% viral infection and replication (FIG. 1. Column B).

### Example 3

### Inhibition of Retroviral Infection and Replication by 6-Amino-4-(4-n-hexyloxy)phenethylaminoquinazoline

293T cells were pretreated with or without 6-Amino-4-(4-n-hexyloxy)phenethylaminoquinazoline (1.5 μM) 30 mins before infection of retrovirus expressing GFP. Two days later, the number of infected cells from two separate experiments was counted, and the quantitative data showed that 6-Amino-4-(4-phenoxy)phenethylaminoquinazoline inhibited about 96% viral infection and replication (FIG. 1. Column C).

### Example 4

### Inhibition of Retroviral Infection and Replication by 6-Amino-4-(4-n-butoxy)phenethylaminoquinazoline

293T cells were pretreated with or without 6-Amino-4-(4-n-butoxy)phenethylaminoquinazoline (1.5 μm) 30 mins before infection of retrovirus expressing GFP. Two days later, the number of infected cells from two separate experiments was counted, and the quantitative data showed that 6-Amino-4-(4-phenoxy)phenethylaminoquinazoline inhibited about 93% viral infection and replication (FIG. 1. Column D).

### Example 5

### Inhibition of Retroviral Infection and Replication by 6-Amino-4-(4-benzyloxy)phenethylaminoquinazoline

293T cells were pretreated with or without 6-Amino-4-(4-benzyloxy)phenethylaminoquinazoline (1.5 μM) 30 mins before infection of retrovirus expressing GFP. Two days later, the number of infected cells from two separate experiments was counted, and the quantitative data showed that 6-Amino-4-(4-phenoxy)phenethylaminoquinazoline inhibited about 90% viral infection (FIG. 1. Column E).

### Example 6

### Inhibition of Retroviral Infection and Replication by 6-Amino-4-[4-(2-pyridylmethyloxy)phenethylaminoquinazoline

293T cells were pretreated with or without 6-Amino-4-[4-(2-pyridylmethyloxy)phenethylaminoquinazoline (1.5 μM) 30 mins before infection of retrovirus expressing GFP. Two days later, the number of infected cells from two separate experiments was counted, and the quantitative data showed that 6-Amino-4-(4-phenoxy)phenethylaminoquinazoline inhibited about 85% viral infection (FIG. 1. Column F).

### Example 7

### Inhibition of Retroviral Infection and Replication by 4-(4-Allyloxy)phenethylamino-6-aminoquinazoline

293T cells were pretreated with or without 4-(4-Allyloxy)phenethylamino-6-aminoquinazoline (1.5 μm) 30 mins before infection of retrovirus expressing GFP. Two days later, the number of infected cells from two separate experiments was counted, and the quantitative data showed that 6-Amino-4-(4-phenoxy)phenethylaminoquinazoline inhibited about 88% viral infection and replication (FIG. 1. Column G).

### Example 8

### Inhibition of Retroviral Infection and Replication by Rocaglamide

293T cells were pretreated with or without rocaglamide (1.5 μm) 30 mins before infection of retrovirus expressing GFP. Two days later, the number of infected cells from two separate experiments was counted, and the quantitative data showed that rocaglamide inhibited 94%±3% viral infection and replication without obvious cell death (FIG. 1. Column H).

### B). The Effect of NF-κB Inhibitors on HIV Infection

### Example 9

### Inhibition of HIV Infection and Replication by Quinazoline Derivatives, Celastrol, Rocaglamide, and Cape

FIG. 2. VSV-G-enveloped HIV-Luciferase viruses were generated as described previously (Li S L, et al., Microbiol Immunol. 2000; 44:1019-1025; Naldini L, et al., Proc Natl Acad Sci USA. 1996; 93:11382-11388). 293T cells were pretreated with 6-Amino-4-(4-phenoxy)phenethylaminoquinazoline (1.5 μM) (Column A), 6-Amino-4-(4-n-pentyloxy)phenethylaminoquinazoline (1.5 μM) ((Column B), 6-Amino-4-(4-n-hexyloxy)phenethylaminoquinazoline (1.5 μM) (Column C), 6-Amino-4-(4-n-butoxy)phenethylaminoquinazoline (1.5 μM) (Column D), 6-Amino-4-(4-benzyloxy)phenethylaminoquinazoline (1.5 μM) (Column E), 6-Amino-4-[4-(2-pyridylmethyloxy)phenethylaminoquinazoline (1.5 μM) (Column F), 4-(4-Allyloxy)phenethylamino-6-aminoquinazoline (3 μM) (Column G), rocaglamide (1.5 μM) (Column H), celastrol (400 nM) (Column I), or cape (5 μg/ml) (Column J), for 30 mins before viral infection. All tested inhibitors showed inhibition of HIV-1 viral infection and replication as indicated by HIV-1-induced Luciferase activity. There were no obvious cell deaths observed with the tested dosages. Data represent the mean±standard deviation of three independent experiments.

### B. The Effect of NF-κB Inhibitors on Cancer Treatment

### Example 10

### The Effect of Quinazoline Derivatives on Cancer Cell Growth

The tested cells include LNCAP human prostate cancer cells, MDA-MB-468 human breast cancer cells, H1299 human lung cancer cells, AGS human gastric cancer cells, HepG2 human liver cancer cells, PANC-1 human pancreatic carcinoma cells, Jurkat human acute T-cell leukemia cells, A431 human epidermoid carcinoma cells, U251 human glioma cells, NBT-II human bladder cancer cells, and Hela human cervical cancer cells. Cultures containing about 4×10⁴ cells were treated with 6-Amino-4-(4-phenoxy)phenethylaminoquinazoline (5 μM), and the viable cells were counted at day 6 using trypan blue dye exclusion assay as described by Chen, et al., in J. Biol. Chem. 273:16700-16709. As shown in Table 1, while untreated cells proliferated and increased cell number by several folds, the cancer cells treated with 6-Amino-4-(4-phenoxy)phenethylaminoquinazoline displayed a range from 10-90% of inhibition of cell growth.

TABLE 1
The effect of 6-Amino-4-(4-phenoxy)phenethylaminoquinazoline
on cancer cell growth.

| | | MDA- | | | | |
|---|---|---|---|---|---|---|
| | LNCAP | MB-468 | H1299 | HT29 | AGS | HepG2 |
| Untreated | 7 ± 0.2 | 6 ± 0.1 | 6.2 ± 0.2 | 3.8 ± 0.4 | 2.4 ± 0.1 | 4.4 ± 0.4 |
| (cell number × | | | | | | |
| 10⁵) | | | | | | |
| Treated | 0.7 ± 0.2 | 5.4 ± 0.1 | 3 ± 0.2 | 1.9 ± 0.1 | 2.1 ± 0.1 | 0.8 ± 0.1 |
| (cell number × | | | | | | |
| 10⁵) | | | | | | |
| % inhibiton | 90 | 10 | 52 | 50 | 10 | 80 |
| | PANC-1 | Jurkat | A431 | U251 | NBT-II | Hela |
| Untreated | 2.2 ± 0.1 | 8.8 ± 0.4 | 5.4 ± 0.4 | 9.1 ± 0.5 | 3 ± 0.1 | 3.6 ± 0.2 |
| (cell number × | | | | | | |
| 10⁵) | | | | | | |
| Treated | 1.9 ± 0.1 | 6.3 ± 0.4 | 3.2 ± 0.2 | 7.2 ± 0.2 | 2.7 ± 0.1 | 1.1 ± 0.1 |
| (cell number × | | | | | | |
| 10⁵) | | | | | | |
| % inhibiton | 14 | 28 | 59 | 20 | 10 | 70 |

The cells listed in Table 1 were also treated with other quinazoline derivatives including 6-Amino-4-(4-n-pentyloxy)phenethylaminoquinazoline, 6-Amino-4-(4-n-hexyloxy)phenethylaminoquinazoline, 6-Amino-4-(4-n-butoxy)phenethylaminoquinazoline, 6-Amino-4-(4-benzyloxy)phenethylaminoquinazoline, 6-Amino-4-[4-(2-pyridylmethyloxy)phenethylaminoquinazoline, 4-(4-Allyloxy)phenethylamino-6-aminoquinazoline, 6-Amino-4-(4-propargyloxy)phenethylaminoquinazoline, 6-Amino-4-(4-ethoxy)phenethylaminoquinazoline, 6-Amino-4-(4-propoxy)phenethylaminoquinazoline, 6-Amino-4-(4-iso-propoxy)phenethylaminoquinazoline. The effects of these compounds on cancer cell growth are comparable to treatment with 6-Amino-4-(4-phenoxy)phenethylaminoquinazoline.

### Example 11

### The Effect of 7-Methoxy-5,11,12-trihydroxy-coumestan on Cancer Cell Growth

7-Methoxy-5,11,12-trihydroxy-coumestan inhibits NF-κB-mediated gene transcription in cells by blocking the phosphorylation and degradation of IκBα. Cultures containing about 4×10⁴ cells were treated with 7-Methoxy-5,11,12-trihydroxy-coumestan (5 μM), and the cells were counted at day 6. In contrasted to untreated cells, 7-Methoxy-5,11,12-trihydroxy-coumestan treatment showed about 51% of inhibition of A431 cell growth, 25% of inhibition of HepG2 cell growth, 31% of inhibition of H1299 cell growth, 29% inhibition of Hela cell growth, and 10% inhibition of MDA-MB-468 cell growth.

### Example 12

### The Effect of 3-hydroxy-24-nor-2-oxo-1(10),3,5,7-friedelatetraen-29-oic Acid (Tripterin; Celastrol, Celastrus scandens) on Cancer Cell Growth and Survival

Cultures containing about 4×10⁴ cells were treated with 3-hydroxy-24-nor-2-oxo-1(10),3,5,7-friedelatetraen-29-oic acid (5 μM), and the viable cells were counted at day 6. Treatment with 3-hydroxy-24-nor-2-oxo-1(10),3,5,7-friedelatetraen-29-oic acid resulted in complete cell apoptosis of a variety of cancer cells including LNCAP cells, MDA-MB-468 cells, H1299 cells, HT29 cells, AGS cells, HepG2 cells, PANC-1 cells, A431 cells, U251 human glioma cells, NBT-II cells, and Hela cells. It caused about 90% of cell apoptosis of Jurkat cells.

### Example 13

### The Effect of (E)3-[(4-Methylphenyl)sulfonyl]-2-propenenitrile (BAY 11-7082) on Cancer Cell Growth and Survival

Cultures containing about 4×10⁴ cells were treated with BAY 11-7082 (5 μM), and the cells were counted at day 6. Treatment with BAY 11-7082 resulted in completely cell apoptosis of a variety of cancer cells including LNCAP cells, H1299 cells, HepG2 cells, PANC-1 cells, A431 cells, and U251 cells, It inhibited about 90% of AGS cell growth, 9-12% of cell growth of MDA-MB-468 cells, NBT-II cells, and Hela cells.

### Example 14

### The Effect of Rocaglamide on Cancer Cell Growth and Survival

Cultures containing about 4×10⁴ cells were treated with rocaglamide (5 μM), and the cells were counted at day 6. Treatment with rocaglamide resulted in completely cell apoptosis of a variety of cancer cells including U251 cells, A431 cells, and MDA-MB-468 cells. It inhibited about 80% of cell growth of H1299 cells, 70% inhibition of LNCAP cell growth, 50% of inhibition of AGS cell growth, 9-40% cell growth of HepG2 cells, PANC-1 cells, NBT-II cells, and Hela cells.

Because NF-κB activity is involved in chronic inflammation and autoimmunity diseases, the chemical compounds should have effect on treatment of chronic inflammation, and autoimmunity diseases.

1. A method of treating a mammal suffering from a viral infection, cancer, chronic inflammation, and autoimmunity diseases comprising the steps of administering to said mammal a chemical targeted to NF-κB and monitoring said mammal to determine state of a viral infection. A method wherein said chemical targeted to said NF-κB is a quinazoline derivative, 3-hydroxy-24-nor-2-oxo-1(10),3,5,7-friedelatetraen-29-oic acid (Tripterin; Celastrol, Celastrus scandens), cape, rocaglamide, 7-Methoxy-5,11,12-trihydroxy-coumestan, (E)3-[(4-Methylphenyl)sulfonyl]-2-propenenitrile (BAY 11-7082), and the compounds with similar structure to them.

2. A methods according to claim 1, wherein a viral infection comprises the infection caused by retrovirus.

3. A methods according to claim 1, wherein a viral infection comprises the infection caused by HIV.

4. A method of treating a mammal suffering from a cancer including lung cancer, breast cancer, skin cancer, liver cancer, cancer in digestive tract, kidney cancer, pancreatic cancer, prostate cancer, brain tumor, human cervical cancer, leukemia, lymphoma, and bladder cancer.

5. A method as in claim 1 wherein said chemical targeted to said NF-κB is a quinazoline derivative represented by the general formula as follows or its pharmaceutically acceptable salt as an active ingredient: 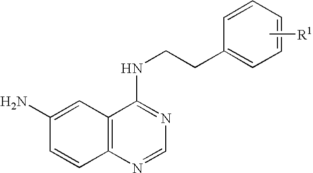
R¹ represents OR², a substituted or unsubstituted aryl, a substituted or unsubstituted heteroaryl, a substituted or unsubstituted alkyl, a substituted or unsubstituted alkenyl, a substituted or unsubstituted alkynyl, the phenooxy group, n-butoxy, n-pentyloxy, n-propoxy, iso-propyl, n-hexyloxy analogues, benzyloxy, pyridylmethoxy, quinolinylmethoxy, allyloxy, propargyloxy, or ethoxy. R² is a substituted or un-substituted alkyl with 1 to 6 carbon, a substituted or un-substituted alkenyl with 2 to 6 carbon, a substituted or un-substituted alkynyl with 2 to 6 carbon, a substituted or un-substituted aryl, substituted or non-substituted heteroaryl, a substituted or un-substituted aralkyl, or a substituted or un-substituted heteroarylalkyl.

6. A method as in claim 5 wherein said chemical is 6-Amino-4-(4-phenoxy)phenethylaminoquinazoline.

7. A method as in claim 5 wherein said chemical is 6-Amino-4-(4-n-pentyloxy)phenethylaminoquinazoline.

8. A method as in claim 5 wherein said chemical is 6-Amino-4-(4-n-hexyloxy)phenethylaminoquinazoline.

9. A method as in claim 5 wherein said chemical is 6-Amino-4-(4-n-butoxy)phenethylaminoquinazoline.

10. A method as in claim 5 wherein said chemical is 6-Amino-4-(4-benzyloxy)phenethylaminoquinazoline.

11. A method as in claim 5 wherein said chemical is 6-Amino-4-[4-(2-pyridylmethyloxy)phenethylaminoquinazoline.

12. A method as in claim 5 wherein said chemical is 4-(4-Allyloxy)phenethylamino-6-aminoquinazoline.

13. A method as in claim 5 wherein said chemical is 6-Amino-4-(4-propargyloxy)phenethylaminoquinazoline.

14. A method as in claim 5 wherein said chemical is 6-Amino-4-(4-ethoxy)phenethylaminoquinazoline.

15. A method as in claim 5 wherein said chemical is 6-Amino-4-(4-propoxy)phenethylaminoquinazoline.

16. A method as in claim 5 wherein said chemical is 6-Amino-4-(4-iso-propoxy)phenethylaminoquinazoline.

17. A method as in claim 1 wherein said chemical is chosen from a group consisting of: 3-hydroxy-24-nor-2-oxo-1(10),3,5,7-friedelatetraen-29-oic acid (Tripterin; Celastrol, Celastrus scandens), cape, rocaglamide, 7-Methoxy-5,11,12-trihydroxy-coumestan, (E)3-[(4-Methylphenyl)sulfonyl]-2-propenenitrile (BAY 11-7082), and the compounds with similar structure to them.

18. A method as in claim 17 wherein said chemical is 3-hydroxy-24-nor-2-oxo-1(10),3,5,7-friedelatetraen-29-oic acid (Tripterin; Celastrol, Celastrus scandens) and its derivatives.

19. A method as in claim 17 wherein said chemical is cape and its derivatives.

20. A method as in claim 17 wherein said chemical is 3-hydroxy-24-nor-2-oxo-1(10),3,5,7-friedelatetraen-29-oic acid (Tripterin; Celastrol, Celastrus scandens) and its derivatives.

21. A method as in claim 17 wherein said chemical is rocaglamide and its derivatives.

22. A method as in claim 17 wherein said chemical is 7-Methoxy-5,11,12-trihydroxy-coumestan, (E)3-[(4-Methylphenyl)sulfonyl]-2-propenenitrile (BAY 11-7082) and its derivatives.

23. A method as in claim 4 wherein said chemical targeted to said NF-κB is a quinazoline derivative represented by the general formula as follows or its pharmaceutically acceptable salt as an active ingredient: 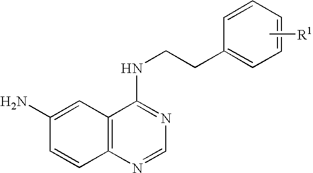
R¹ represents OR², a substituted or unsubstituted aryl, a substituted or unsubstituted heteroaryl, a substituted or unsubstituted alkyl, a substituted or unsubstituted alkenyl, a substituted or unsubstituted alkynyl, the phenooxy group, n-butoxy, n-pentyloxy, n-propoxy, iso-propyl, n-hexyloxy analogues, benzyloxy, pyridylmethoxy, quinolinylmethoxy, allyloxy, propargyloxy, or ethoxy. R² is a substituted or un-substituted alkyl with 1 to 6 carbon, a substituted or un-substituted alkenyl with 2 to 6 carbon, a substituted or un-substituted alkynyl with 2 to 6 carbon, a substituted or un-substituted aryl, substituted or non-substituted heteroaryl, a substituted or un-substituted aralkyl, or a substituted or un-substituted heteroarylalkyl.

24. A method as in claim 4 wherein said chemical is chosen from a group consisting of: 3-hydroxy-24-nor-2-oxo-1(10),3,5,7-friedelatetraen-29-oic acid (Tripterin; Celastrol, Celastrus scandens), cape, rocaglamide, 7-Methoxy-5,11,12-trihydroxy-coumestan, (E)3-[(4-Methylphenyl)sulfonyl]-2-propenenitrile (BAY 11-7082), and the compounds with similar structure to them.
